(19) United States
(12) Patent Application Publication
(10) Pub. No.: US 2001/0045027 A1
(43) Pub. Date: Nov. 29, 2001

(54) Perfected insole for footwear

(76) Inventor: Miguel Angel Sanchez-Osorio Esteban, Barcelona (ES)

Correspondence Address: CROWELL & MORING LLP, INTELLECTUAL PROPERTY GROUP, P.O. BOX 14300, WASHINGTON, DC, 20044-4300, US

(21) Appl. No.: 09/783,532

(22) Filed: Feb. 15, 2001

(30) Foreign Application Priority Data
May 23, 2000 (ES) 200001363

(51) Int. Cl. A43B 7/14; A61F 5/14

(52) U.S. Cl. 36/43; 36/166; 36/180

(57) ABSTRACT

A perfected insole for footwear of the type formed by a flexible laminate body cut in the form of the interior surface of the sole or base of the footwear. The laminate body corresponds to an end zone which has a modified horizontal direction. The upper face of the laminate body has a central raised zone portion of the same width as the insole. That extends laterally in a longitudinal direction on the interior part of the area of the arch of the foot to the heel part, with the central raised zone covering the tarsus and metatarsus area.

### BACKGROUND OF THE INVENTION

This application claims the priority of Spanish Application 200001363/1, filed May 23, 2000, the disclosures of which are expressly incorporated by reference herein.

The present invention relates to a perfected insole for footwear which is made to correct and rehabilitate walking and running problems.

There are currently people who have problems and pathologies with respect to walking correctly. These problems increase generally over time in a qualitative and quantitative manner, and in the worst cases may cause grave degeneration of articulation functions.

In an attempt to avoid this, insoles, traditional treatments of rehabilitation, physiotherapy, and even recovery static bicycles have been used. Generally all these systems do not solve the cause of the problem, for example, walking badly even though some of the pain and degeneration caused by the problem may be alleviated.

### SUMMARY OF THE INVENTION

An object of the present invention is to provide an insole whose constitution and design, carries out the corrections according to the needs of the person, with the same treatment.

Therefore, the insole of the present invention constitutes the basis for the above-mentioned treatment system.

According to the present invention, the insole is formed by a noticeably laminate body on which the horizontal direction of the front part is modified, corresponding to the toe end and the zone next to the sole. The central part of the insole has on its upper face, which is the visible one, a raised part that extends from the interior zone corresponding to the sole or wide area, down to the heel. The raised zone is not uniform or regular, since the thickest part is located on the interior lateral part of the insole. Once placed inside the shoe, the insole can be used by the person requiring treatment. The insole forms a real and effective support for the sole of the foot, against the raised part of the insole, so that this zone is impressed by deformation for the correction to be made later to the insole to correct the defects of the biomechanics of the foot itself.

Naturally such defects that appear in the upper part of the insole means that as a consequence of adapting to the food a deformation appears in the front of the insole due to the pressure of the defect itself or of the dynamic problem.

Once the insole is corrected and adapted to the sole of the foot, the person carries out dynamic and sport exercises when wearing the footwear with the corrected insole during the first phase of treatment, and later after a period of time and use, checks are carried out for possible corrections that may be necessary with use.

### BRIEF DESCRIPTION OF THE DRAWINGS

Other objects, advantages and novel features of the present invention will become apparent from the following detailed description of the invention when considered in conjunction with the accompanying drawings wherein:

FIG. 1 is a plane view of the upperside of an insole according to the present invention;

FIG. 2 is a sectional view of the insole of FIG. 1;

FIG. 3 is a plane view of the underside of the insole of FIG. 1.

FIG. 4 is a end sectional view of the insole of the FIGS. 1 and 3;

FIG. 5 is a plane view of another embodiment of the insole of the invention;

FIG. 6 is a side sectional view of the insole of FIG. 5; and

FIG. 7 is a side view of the insole of FIG. 5.

### DETAILED DESCRIPTION OF THE DRAWINGS

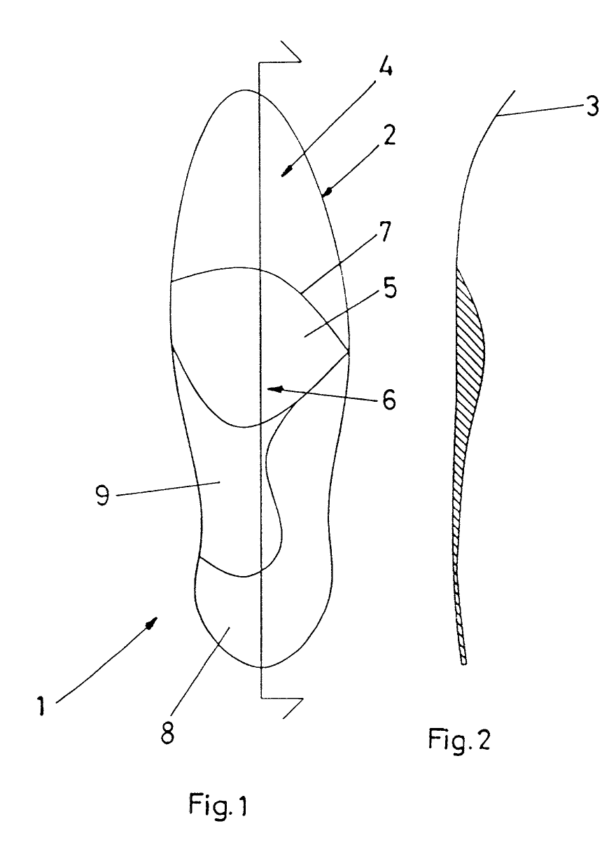

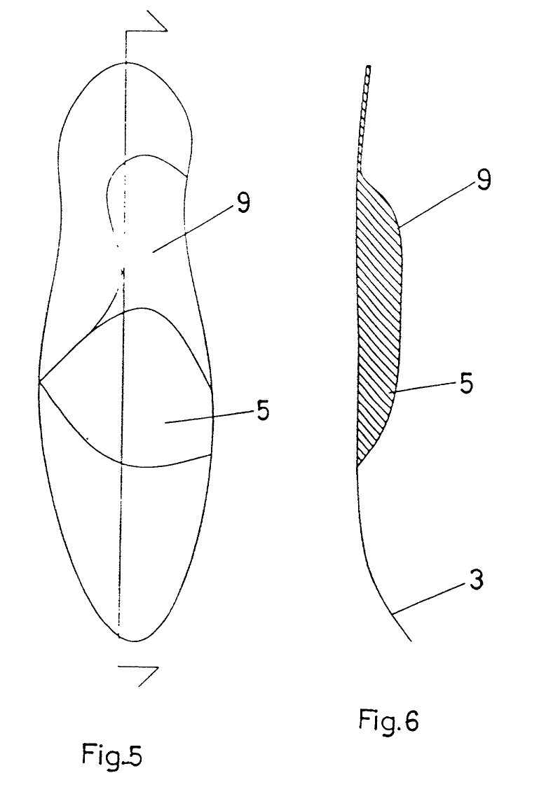

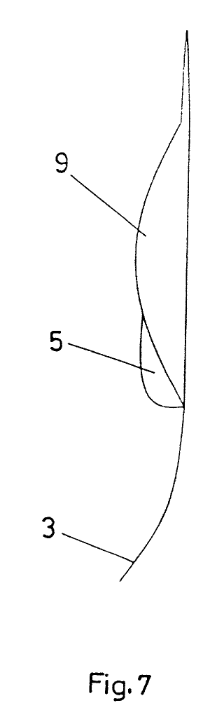

The insole shown in FIGS. 1-4 is made of a laminated body 2. In the area of the toe, horizontal direction 4 of the insole is modified and a zone 5 appears on the upper face with a raised part 6 that extends from the front or wide zone 7 to the heel.

This embodiment has two defined parts as viewed from above, namely one of greater width 8 and another of reduced width and greater length 9.

The raised part coincides with the tarsus and metatarsus.

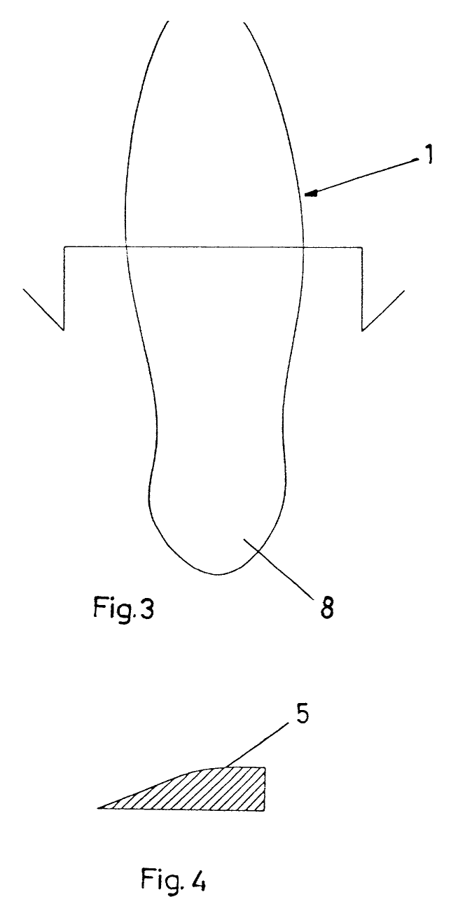

The zone of greatest thickness of the insole corresponds to the interior lateral area of the foot, FIGS. 5 and 6, while that of least thickness corresponds to the exterior lateral area as can be seen in FIGS. 1 and 2.

The central part of the insole occupies the width of the top of the same, while the part corresponding to the arch and heel area, being of irregular shape, is elongated and runs according to the ideal longitudinal axis of the insole.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. Perfected sole for footwear; of the type formed by a flexible laminate body cut to a shape the same as the internal surface of the sole or base of the footwear; characterized because the laminate body corresponds to an end zone, corresponding to the toe, which has its horizontal direction modified, while the upper face of this laminate body has a raised zone in its central part, of the same width as the insole, extending laterally in a longitudinal direction on the interior part of the area of the arch of the foot towards the heel area, covering the raised area of the tarsus and metatarsus.

2. Insole according to claim 1, characterized because the zone of greatest thickness corresponds to the lateral interior zone.
